(19) United States
(12) Patent Application Publication
(10) Pub. No.: US 2003/0072411 A1
(43) Pub. Date: Apr. 17, 2003

(54) Beam parameter display on console screen

(76) Inventor: Donald E. Welsh, Walnut Creek, CA (US)

Correspondence Address: Siemens Corporation, Att: Elsa Keller, Legal Administrator, Intellectual Property Department, 186 Wood Avenue South, Iselin, NJ, 08830, US

(21) Appl. No.: 09/978,807

(22) Filed: Oct. 17, 2001

(51) Int. Cl. A61N 5/10

(52) U.S. Cl. 378/65; 378/108

(57) ABSTRACT

A system, method, apparatus, and means for tuning a radiation therapy device using a treatment processing unit coupled to said radiation therapy device includes selecting an operational parameter of the radiation therapy device. A signal converter is configured to receive an output signal from the radiation therapy device, the output signal depending on the operational parameter. The operational parameter is then varied while displaying the output signal. In some embodiments, the operational parameter is continually varied until the output signal reaches a desired value.

### BACKGROUND OF THE INVENTION

1. Field of the Invention

The present invention relates generally to radiation therapy devices, and more particularly, to systems and methods for the maintenance, tuning, and servicing of such devices.

2. Description of the Related Art

Radiation therapy is commonly used in the treatment of diseases and is quite effective in destroying cancer cells. The devices used to deliver therapeutic radiation in radiation therapies are complex devices designed to direct a radiation beam at a tumor in a patient to deliver a predetermined dose of therapeutic radiation to the tumor according to an established treatment plan. This is typically accomplished using a radiation therapy device such as the device described in U.S. Pat. No. 5,668,847 issued Sep. 16, 1997 to Hernandez, the contents of which are incorporated herein for all purposes.

Radiation therapy devices are complex and expensive devices which are commonly used by hospitals and treatment centers to treat patients, often on a tight and demanding treatment schedule. The overall reliability of radiation therapy devices is an important concern to users of the devices and to patients. Failure of the device is potentially devastating to both the hospital (in terms of revenue, scheduling, and patient care) as well as to patients who have a real and pressing need for uninterrupted treatment.

Further, the radiation therapy treatment of tumors requires a high degree of accuracy. The radiotherapy treatment of tumors involves three-dimensional treatment volumes which typically include segments of normal, healthy tissue and organs. Healthy tissue and organs are often in the treatment path of the radiation beam. This complicates treatment, because the healthy tissue and organs must be taken into account when delivering a dose of radiation to the tumor. While there is a need to minimize damage to healthy tissue and organs, there is an equally important need to ensure that the tumor receives an adequately high dose of radiation. Cure rates for many tumors are a sensitive function of the dose they receive. Therefore, it is important to closely match the radiation beam's shape and effects with the shape and volume of the tumor being treated. Accurate control and manipulation of the beam is needed to ensure proper treatment of patients and that healthy tissue and organs are not damaged during treatment.

One aspect of radiation therapy devices which can degrade over time is the accuracy and efficiency of the beam delivered. As a result, manufacturers and maintainers of radiation therapy devices commonly perform routine servicing of radiation therapy devices to ensure the beam (whether it be an X-ray, photon, or other type of beam) is properly adjusted or “peaked”. This process of beam peaking is typically performed on a regular basis, often in conjunction with scheduled routine maintenance, and ensures that the radiation therapy device is accurately delivering a beam of a desired intensity.

Currently, beams are peaked by attaching two oscilloscopes to one or more beam feed cables emerging from a radiation therapy room. The oscilloscopes are often set up near or next to an operator console so that the oscilloscope screens can be read by the service technician while manipulating controls of the operator console. This process is expensive and time consuming, as the beam can be peaked only by a technician who brings oscilloscopes and other measurement devices with him on a service call. Hooking up the measurement equipment can be time consuming and subject to errors which can lead to inconsistent results.

It would be desirable to provide a system and method which allows radiation beams generated by a radiation therapy device to be peaked, verified, and otherwise maintained without the need for specialized external equipment and without the need to make additional connections during a service call. It would further be desirable to provide a system and method which allows operators of radiation therapy devices to readily verify beam performance characteristics to determine when tuning or other maintenance is required.

### SUMMARY OF THE INVENTION

To alleviate the problems inherent in the prior art, and to allow efficient and ready tuning, maintenance, and diagnostic analysis of radiation therapy devices, embodiments of the present invention provide a method, apparatus, and means for displaying beam parameters on a console screen.

According to one embodiment of the present invention, a system, method, apparatus, and means for tuning a radiation therapy device using a treatment processing unit coupled to said radiation therapy device includes selecting an operational parameter of the radiation therapy device. A signal converter is configured to receive an output signal from the radiation therapy device, the output signal depending on the operational parameter. The operational parameter is then varied while displaying the output signal. In some embodiments, the operational parameter is continually varied until the output signal reaches a desired value.

According to some embodiments of the present invention, the operational parameter includes at least one of: a dose rate, an energy level, an operational mode, an input power frequency, and an input to an electromagnet. According to some embodiments of the present invention, the output signal includes at least one of: a beam current, an RF reflected power signal, a pulse current, a dose pulse signal, an injection current, and an input power frequency.

According to some embodiments of the present invention, both the operational parameter and the output signal are displayed on a single display device of a treatment processing unit.

The present invention is not limited to the disclosed preferred embodiments, however, as those skilled in the art can readily adapt the teachings of the present invention to create other embodiments and applications.

### BRIEF DESCRIPTION OF THE DRAWINGS

The exact nature of this invention, as well as its objects and advantages, will become readily apparent from consideration of the following specification as illustrated in the accompanying drawings, in which like reference numerals designate like parts throughout the figures thereof, and wherein:

FIG. 1 is diagram illustrating a radiation therapy device and treatment processing unit;

FIG. 2 is a block diagram illustrating portions of the radiation therapy device and treatment processing unit of FIG. 1 according to one embodiment of the present invention;

FIG. 3 is a flow diagram illustrating a process for the tuning and verification of the beam generated by the system of FIG. 1 according to one embodiment of the present invention; and

FIGS. 4A-B illustrate example display screens from a treatment processing unit of FIG. 1 according to one embodiment of the present invention.

### DETAILED DESCRIPTION

The following description is provided to enable any person skilled in the art to make and use the invention and sets forth the best modes contemplated by the inventor for carrying out the invention. Various modifications, however, will remain readily apparent to those skilled in the art.

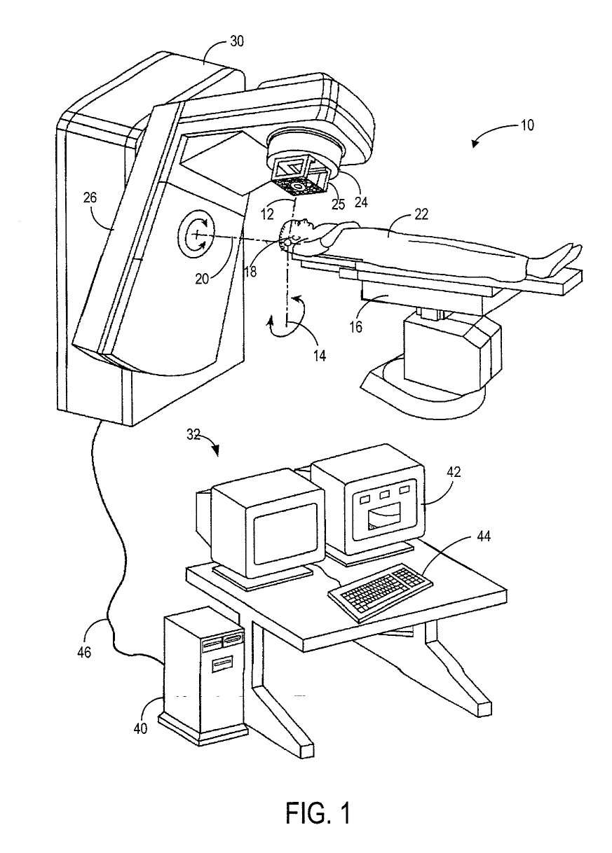

Turning now to the drawings and, with particular attention to FIG. 1, a radiation therapy device 10 pursuant to embodiments of the present invention is shown. According to one embodiment of the present invention, radiation therapy device 10 includes a beam shielding device (not shown) within a treatment head 24, a control unit in a housing 30 and a treatment unit 32. An accessory tray 25 is mounted to an exterior of treatment head 24. Accessory tray 25, in one embodiment, is configured to receive and securely hold attachments used during the course of treatment planning and treatment (such as, for example, reticles, wedges, or the like).

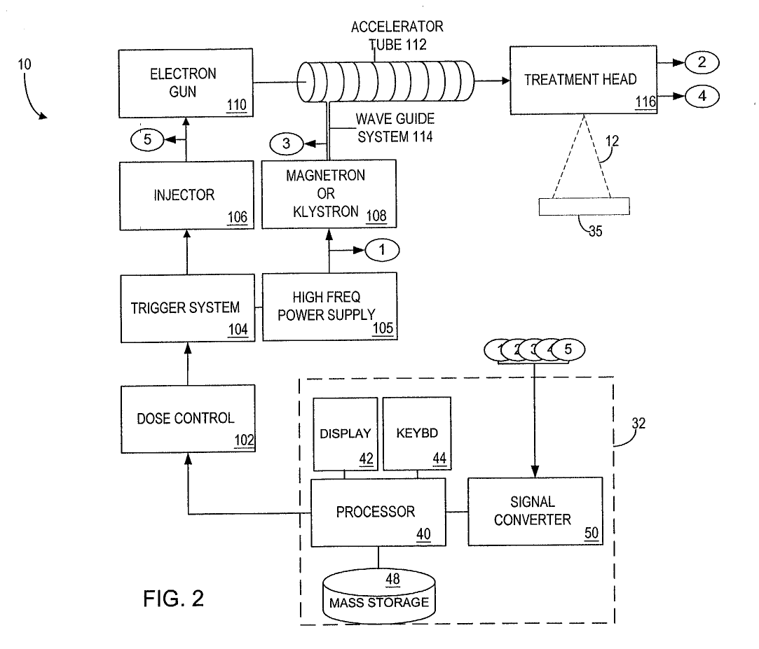

Radiation therapy device 10 includes a gantry 26 which can be swiveled around a horizontal axis of rotation 20 in the course of a therapeutic treatment. Treatment head 24 is fastened to a projection of the gantry 26. A linear accelerator (shown as item 112 in FIG. 2) is located inside gantry 26 to generate the high energy radiation required for the therapy. The axis of the radiation bundle emitted from the linear accelerator and the gantry 26 is designated by beam path 12. Electron, photon or any other detectable radiation can be used for the therapy. Embodiments of the present invention permit an operator or service technician to quickly and efficiently optimize and/or verify the beam generated by radiation therapy device 10 without requiring the installation and connection of external equipment. As a result, the beam generated by radiation therapy device 10 may be tuned, verified, and analyzed on a more frequent basis, ensuring that treatments delivered using the device are capable of achieving a high degree of accuracy.

Radiation therapy device 10 also includes a central treatment processing unit 32 which is typically located apart from radiation therapy device 10. Radiation therapy device 10 is normally located in a different room to protect an operator of treatment processing unit 32 from radiation. Treatment processing unit 32 is coupled to radiation therapy device 10 via one or more beam signal cables 46 which are typically routed from a treatment room to a separate or shielded control room.

Treatment processing unit 32 includes a processor 40 in communication with a display 42 (including one or more visual display units or monitor) and an input device such as a keyboard 44. Data can also be input through data carriers such as data storage devices or a verification and recording or automatic setup system. More than one treatment processing unit 32, processor 40, keyboard 44 and/or display 42 may be provided to control radiation therapy device 10.

Treatment processing unit 32 is typically operated by a therapist who administers actual delivery of radiation treatment as prescribed by an oncologist. A therapist typically operates treatment processing unit 32 by using keyboard 44 or other input device. The therapist enters data defining the radiation dose to be delivered to the patient, for example, according to the prescription of the oncologist. The program can also be input via another input device, such as a data storage device. Various data can be displayed before and during the treatment on the screen of display 42.

In some embodiments, treatment processing unit 32 may be operated in a service, maintenance or diagnostic mode under the control of the therapist, technician or operator (together, generically referred to herein as an “operator” of treatment processing unit 32). According to some embodiments, an operator controlling treatment processing unit 32 in a service, maintenance or diagnostic mode may perform service, maintenance and/or diagnostic functions such as beam peaking or tuning of radiation therapy device 10.

Embodiments of the present invention permit efficient and cost effective adjustment, tuning and verification of beams generated by radiation therapy device 10. Unlike previous tuning systems, embodiments of the present invention permit beam peaking and analysis without the need to connect external oscilloscopes and measuring devices thereby reducing equipment cost, maintenance time, and errors. Further, as will be described further below, embodiments of the present invention permit an operator or technician to perform beam peaking based on the real time presentation of signal data on display 42 along with input and control data. The result is a system and method which provides improved maintenance efficiency and accuracy.

Referring now to FIG. 2, a block diagram is shown depicting portions of a radiation therapy device 10 and treatment unit 32 according to one embodiment of the present invention. In particular, treatment delivery elements of a radiation therapy device are shown, which may be configured in radiation therapy device 10 and treatment processing unit 32 as depicted in FIG. 1.

Radiation therapy device 10 includes an accelerator tube 112 which is used to accelerate electrons generated by an electron gun 110 through a treatment head 116. The resulting radiation bundle or beam 12 is directed from treatment head 116 toward a target zone on a patient (not shown). In some embodiments, a detector 35 is positioned along a path of beam 12 to measure characteristics of the beam. For example, detector 35 may be a dosimetry device used to accurately measure a dose delivered by beam 12. Suitable devices are offered by, for example, Scanditronix Wellhofer of Bartell, Tenn.

Other components of radiation therapy device 10 include a dose control unit 102 which receives control signals from treatment processing unit 32 and which controls a trigger system 104 to generate injector trigger signals and supply them to injector 106. Trigger or other control signals are also provided to control a high frequency source 108 (such as a magnetron or klystron). Based on the injector trigger signals, injector 106 generates injector pulses which are fed to electron gun 110 which in turn generates electron bunches which are input to accelerator tube 112 for acceleration.

A high frequency power supply 105 is coupled to provide high frequency switched power to drive high frequency source 108. Power supply 105 may be any of a number of different types of designs used in the art, including, for example, a device using a pulse forming network and a switching device allowing the controlled generation of high frequency power to drive a load such as a klystron or magnetron.

High frequency source 108 is coupled to a wave guide system 114 for providing high frequency signals to generate an electromagnetic field in accelerator tube 112. The electrons injected by injector 106 and emitted by electron gun 110 are accelerated by this electromagnetic field in accelerator tube 112 and exit at the treatment head as beam 12. Those skilled in the art appreciate that a typical radiation therapy device 10 includes a number of other elements used to generate, control and deliver beam 12 which are not depicted here.

Components of treatment processing unit 32 depicted in FIG. 2 include a processor 40, operatively coupled to a keyboard 44 for receiving control inputs and a display 42 for displaying data to an operator or service technician. Processor is also operatively coupled to a signal converter 50 for receiving representations of signals from various components of radiation therapy device 10. Processor 40 may be one or more microprocessors configured to control and manipulate radiation therapy device 10. Although a single processor 40 is depicted in FIG. 2, those skilled in the art will appreciate that the functions described herein may be accomplished using one or more computing devices operating together or independently. Those skilled in the art will also appreciate that any suitable general purpose or specially programmed computer may be used to achieve the functionality described herein.

As depicted, processor 40 is coupled to display 42 and keyboard 44. In some embodiments, processor 40 may be coupled to additional input devices such as, for example, additional keyboards, a mouse or other pointing device, a microphone, a knob or a switch (including an electronic representation of a knob or a switch), an infrared port, a docking station, and/or a touch screen. In some embodiments, processor 40 may be coupled to additional output devices such as, for example, additional displays, one or more speakers, and/or printers.

During a treatment, treatment processing unit 32 is typically controlled by an operator who administers the delivery of a radiation treatment as prescribed by an oncologist. By interacting with keyboard 44 and display 42, the operator enters data that defines the radiation to be delivered to a patient. According to embodiments of the present invention, during maintenance, servicing, or diagnostics of radiation therapy device 10, treatment processing unit 32 is controlled by an operator who manipulates inputs to radiation therapy device 10 while monitoring effects of changes on signal data presented on display 42. By interacting with keyboard 44 and display 42, the operator enters data varying control parameters of radiation therapy device 10 (e.g., beam intensity, beam type, input power frequency, etc.) while monitoring the effects of the variations on output parameters (e.g., the output beam current, the input pulse current, the RF reflected power, etc.). A variety of different types of maintenance, service and diagnostics may be performed in this manner. One example maintenance procedure is beam peaking. Those skilled in the art, upon reading this disclosure, will recognize that embodiments of the present invention may be used to conduct other types of maintenance, service and diagnostic procedures.

Processor 40 is also in communication with a mass storage device 48. Mass storage device 48 comprises an appropriate combination of magnetic, optical and/or semiconductor memory, and may include, for example, Random Access Memory (RAM), Read-Only Memory (ROM), a compact disc and/or a hard disk. Processor 40 and mass storage device 48 may each be, for example: (i) located entirely within a single computer or other computing device; or (ii) connected to each other by a remote communication medium, such as a serial port cable, telephone line or radio frequency transceiver. In one embodiment, processor 40 may comprise one or more computers that are connected to a remote server computer for maintaining databases.

Mass storage device 48 stores data used and generated during the operation of the radiation therapy device including, for example, treatment data as defined by an oncologist for a particular patient. This treatment data is generated, for example, using a treatment planning system (not shown) which may include manual and computerized inputs to determine a beam shape prior to treatment of a patient. Treatment planning systems are typically used to define and simulate a beam shape required to deliver an appropriate therapeutic dose of radiation to treatment zone 18.

Mass storage device 48 may also store other information and programs used to operate radiation therapy device 10. For example, mass storage device 48 may store one or more interlock libraries, each defining one or more interlocks to be used in the operation of radiation therapy device 10 in a particular operation mode (e.g., different interlocks may be used depending on whether the treatment uses primary electrons, primary photons, or mixture of primary electrons and primary photons). According to some embodiments of the present invention, mass storage device 48 also stores data and programs used to perform maintenance and tuning, such as beam peaking, pursuant to embodiments of the present invention. For example, mass storage device 48 may store signal data captured by signal converter 50.

Embodiments of the present invention include one or more signal converters 50 coupled to processor 40. Signal converter 50 is coupled to receive one or more signals from radiation therapy device 10 and is configured to generate digital representations of each of the signals. The digital representations of the signals are provided to processor 40 for further manipulation and/or for output to a service technician or operator via display 42. Any of a number of different signal converters 50 may be used in embodiments of the present invention. For example, one or more of the CompuScope® analog input cards offered by Gage Applied, Inc. of Lachine Canada may be used. In some embodiments, several signal converters 50 are used to capture several separate signal channels. In some embodiments, mass storage device 48 stores signal data received via signal converter 50 and also stores software used to manipulate and present signal data on display 42. For example, mass storage device 48 may store the GageScope® Oscilloscope software offered by Gage Applied, Inc. of Canada which may be used in conjunction with processor 40 and data captured by signal converter 50 to present signal data on display 42.

In some embodiments, signal converter 50 is coupled to beam signal cables 46 to receive various operational signals from radiation therapy device 10. In an embodiment where radiation therapy device 10 is configured to deliver both photon and X-ray radiation, five signals may be input to signal converter 50 (labeled on FIG. 2 as items 1-5). Those skilled in the art will recognize that other signals may also be input to signal converter 50 to perform other analysis and tuning of radiation therapy device 10. In one embodiment, receipt of these different signals involves the use of several different analog input cards, each capturing several different input signals.

A pulse current 1 is measured from an input to a high frequency source 108 such as a magnetron or klystron. A beam current 2 is measured from a target that produces photons in the treatment head. An RF reflected power signal 3 is detected from an output of the high frequency source 108 (e.g., at the output of either a magnetron or klystron). In some embodiments, the RF reflected power signal 3 is detected at a four port circulator at the load. A dose pulse 4 is measured from an ion chamber positioned within treatment head 116. Two different ion chambers may be provided in the treatment head, one used when the device is operated in a photon mode, and one used when the device is operated in an X-ray mode. Dose pulse 4 is measured from either of the ion chambers based on the operating mode. An injection current 5 is measured by sensing the current entering the injector gun 106. Those skilled in the art will recognize that other signals may be detected and analyzed to allow an operator or technician to perform maintenance or analysis of the quality and accuracy of beam 12.

Processor 40 is also operatively coupled to various elements of radiation therapy device 10. This connection may be via, for example, beam signal cables 46. For example, processor 40 may be operatively coupled to control units (not shown) of radiation therapy device 10 including, for example, control units utilized to manipulate gantry 26, table 16, etc. These devices are controlled by processor 40 to place a patient in a proper position to receive treatment from the radiation therapy device. In some embodiments, gantry 26 and/or table 16 may be repositioned during treatment to deliver a prescribed dose of radiation.

Processor 40 is also operatively coupled to a dose control unit 50 which includes a dosimetry controller and which is designed to cause elements of radiation therapy device 10 to generate a desired beam 12 having desired isodose curves. Processor 40 is further operatively coupled to control various inputs and operational variables of radiation therapy device 10, such as, for example, the energy to be delivered, the dose to be delivered, and the power input to the radiation therapy device 10 (e.g., from a pulse forming network or other power supply, not shown). Pursuant to embodiments of the present invention, each of these operational variables may be manipulated under the control of treatment processing unit 32 while viewing the effects the changed variables have on output characteristics detected by signal converter 50. This allows ready and efficient analysis, tuning, and maintenance of radiation therapy device 10. In some embodiments, operational variables of radiation therapy device 10 are be varied by an operator interacting with control software stored at treatment processing unit 32. According to embodiments of the present invention, the effects of changes in operational variables are detected and presented to the operator at treatment processing unit 32. According to embodiments of the present invention, the accuracy and control of these operational variables may be tuned or verified by allowing the operator to view and manipulate beam effects.

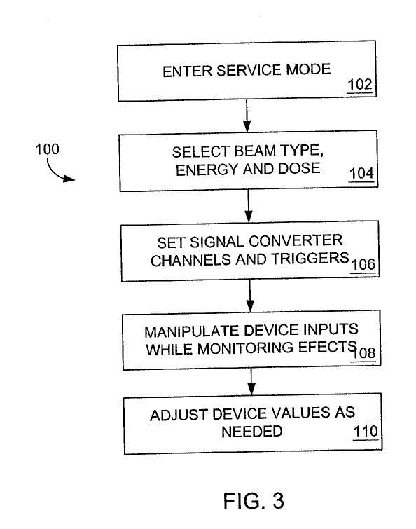

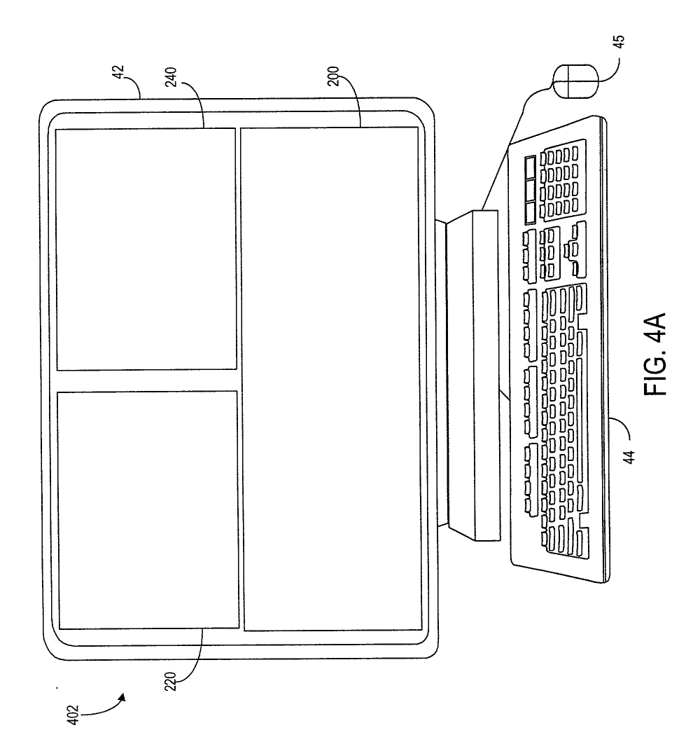

Referring now to FIG. 3, a process 100 for tuning radiation therapy device 10 is shown. The flow chart in FIG. 3 does not imply a fixed order to the steps, and embodiments of the present invention can be practiced in any order that is practicable. The method shown in FIG. 3 may be performed, for example, by the treatment processing unit 32 operated in conjunction with radiation therapy device 10. In one embodiment, process 100 may be orchestrated through the use of a set of menu screens displayed on display 42, with which an operator may interact to conduct process 100. Similar processes may be established and conducted for other maintenance, diagnostic, or servicing procedures.

Processing begins at 102 where an operator, technician, or other individual (for simplicity, referred to as the “operator”) controls treatment processing unit 32 to enter a service mode or other mode which allows treatment processing unit 32 to be used to verify or adjust settings of radiation therapy device 10. This step 102 is optional, however, in some situations it is desirable to require use of a service mode separate from an operational mode so that only trained service personnel are able to perform maintenance and tuning as described herein. In some embodiments, processing at 102 may require use of a special key or code to enter a mode allowing the operator to interact with treatment processing unit 32 to conduct process 100.

Processing continues at 104 where the operator manipulates treatment processing unit 32 to select a beam type (if radiation therapy device 10 supports more than one beam type), a desired energy, and a desired dose. As an example where the operator intends to perform beam peaking and tuning of the X-Ray mode of a radiation therapy device, processing at 104 may involve the selection of X-Ray as the beam type and 6 MV as the desired energy and 200 Monitor Units (MU)/Minute as the desired dose. Process 100 may be repeated for various energies and doses to tune radiation therapy device 10.

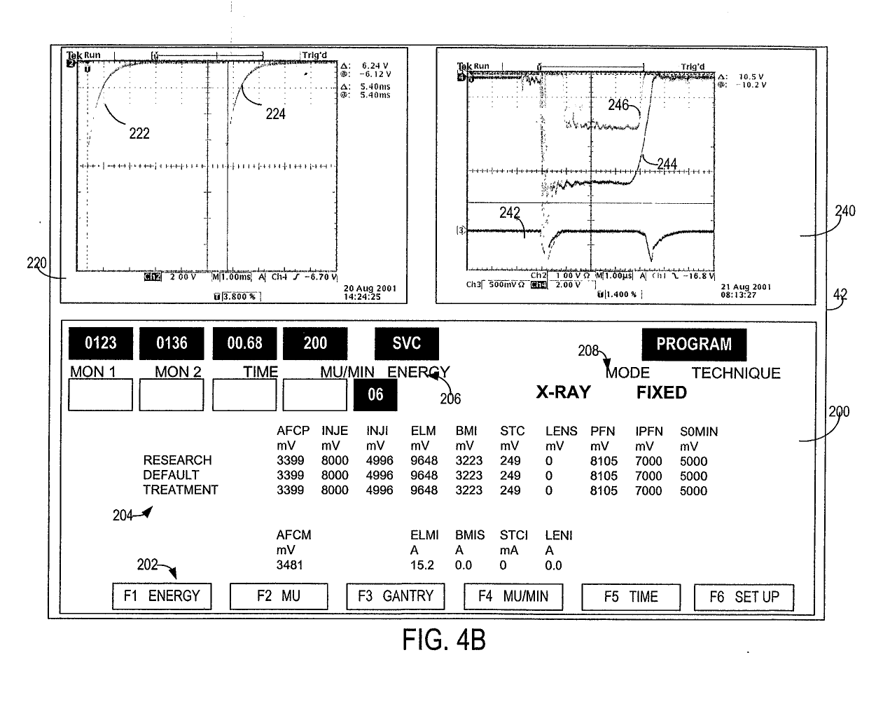

Processing continues at 106 where the operator manipulates treatment processing unit 32 to set signal converter channels and triggers. In one embodiment, this may include running software associated with signal converter 50 which allows channels and triggers to be set and otherwise configured. According to some embodiments of the present invention, processing at 106 and processing at 104 may be accomplished using a split screen on display 42 of treatment processing unit 32 (with one screen showing values and options associated inputs and control of radiation therapy device 10 and one or more screens showing values and options associated with the signals received via signal converter 50). In some embodiments, processing at 106 and 104 may be accomplished by switching between display screens of display 42 of treatment processing unit 32. In some embodiments, treatment processing unit 32 may include multiple displays 42, each of which may display some or all of the information used to perform processing at 104 and 106. One example arrangement of display 42 is depicted in FIG. 4 which will be discussed further below.

A variety of different combinations of signal converter channels and triggers may be utilized to tune or monitor radiation therapy device 10. As an example where an operator is tuning the X-Ray mode of the device, two channel settings may be set, including a first channel receiving the following signals: beam current 2, RF reflected 3, and pulse current 1. A second channel may also be set up, receiving: dose pulse 4, and injection current 5. Triggers for each may be established as is known in the art. According to some embodiments, signal displays for each channel are displayed on display 42 of treatment processing unit 32 along with information used at 104. In some embodiments, the information is displayed in multiple windows on display 42. In this manner, the operator may change radiation therapy device 10 inputs while monitoring effects of those inputs on signals received by signal converter 50.

Once signal converter channels and triggers have been set, processing continues to 108 where the operator interacts with treatment processing unit 32 to manipulate device inputs while monitoring effects. As an example, a radiation beam may be “peaked” by monitoring dose pulse 4 while increasing the frequency of the high frequency power signal supplied to magnetron or klystron 108. As the frequency of the power signal is increased to its maximum value, the shape of the beam current 2 signal is monitored to identify the frequency at which the beam current 2 signal is relatively flat. At the same time, the operator may also manipulate input signals to vary inputs to the electromagnet of the radiation therapy device 10 to produce a relatively clean RF reflected power signal 3. The point at which the RF reflected power signal 3 and the beam current signal 2 have a clean and flat shape is the point where the X-Ray beam is peaked. In some embodiments, processing at 108 begins in a closed loop state. To determine if the beam is properly peaked, processing at 108 may conclude by switching to an open loop state to determine if the dose rate changes. If the dose rate value changes between the closed loop state and the open loop state, the beam may not be properly peaked or the dosimetry system may not be fully calibrated.

If the dose rate changes between closed and open loop, processing may continue by manipulating the frequency of the power signal while radiation therapy device 10 is in the open loop state, again attempting to achieve a clean and flat beam current signal 2 and RF reflected power signal 3. Once these clean and flat signals are achieved, the injection current 5 is reduced or increased (within safe operating limits) to achieve the desired dose rate. The operator may then toggle radiation therapy device 10 between open and closed loop modes to verify that the dose rate is the same in both modes.

Processing then continues to 110 where the operator records any adjustments to device values. For example, the input values which resulted in the peaked beam for a given dose rate are recorded and used to update dosimetry data for radiation therapy device 10. Subsequent uses of the device will enjoy greater accuracy as a result.

Process 100 may be repeated on an regular or as-needed basis to maintain radiation therapy device 10 in an accurate and reliable operating condition.

Referring now to FIG. 4A, an example user interface is shown. Display 42 may be used to display a variety of information used to maintain radiation therapy device 10. In some embodiments, display 42 may display a number of different windows of information. As shown in FIG. 4A, display 42 presents data in three different windows, including a primary window 200 which is used to present control data and information about the operation of radiation therapy device 10. Two secondary windows 220 and 240 are also depicted, which may be used to present data representing signal information captured by signal converter 50. Other combinations and variations of windows may be used to present data and information used to maintain and tune radiation therapy device 10. An operator may maneuver between windows using keyboard 44 or other input devices (such as mouse 45) as is known in the art. According to some embodiments of the present invention, data and information needed to tune and maintain radiation therapy device 10 is presented without requiring the hookup of external measurement devices such as oscilloscopes or the like.

A user interface presenting example data and information used to tune and maintain radiation therapy device 10 is shown in FIG. 4B. As depicted, the user interface presented on display 42 includes three windows, 200, 220 and 240. Primary window 200 presents control data and information about the operation of radiation therapy device 10, including preset data 206 which entered by an operator or which is entered by processor 40 in response to operator input. Function keys 202 are included along a bottom of window 200, and machine status information 208 is presented along a top of window 200. Control information 204 is presented in a middle portion of window 200, and reflects variable data which change in response to field parameters selected by the operator. Other types and presentations of data may also be used. An operator attempting to tune radiation therapy device 10 may input and view field parameters via primary window 200.

Window 220 depicts signal data captured by signal converter 50 and converted to image data using software operated by processor 40. In particular, in the example data shown in FIG. 4B, window 220 is used to display pulse repetition time information (captured by signal converter 50 from radiation therapy device 10). An operator tuning radiation therapy device 10 may view the data in window 220 to determine when the frequency of the power signal input to high frequency source 108 reaches an appropriate large value.

Window 240 depicts signal data captured by signal converter 50 and converted to image data using software operated by processor 40. In particular, in the example data shown in FIG. 4B, window 240 is used to display three different channels of data including beam current 2 (represented as signal 246), RF reflected power 3 (represented as signal 242) and pulse current 1 (represented as signal 244). As described above in conjunction with FIG. 3, an operator manipulates input data and operational parameters (e.g., via window 200) while viewing the output effects (e.g., via windows 220 and 240) until a desired output effect is reached. In an example tuning process where an X-Ray beam is being peaked, the operator will manipulate the input power frequency and view the shape of the beam current signal 246. By allowing the operator to enter information and view output effects via treatment processing unit 32, maintenance and tuning operations are simplified, allowing more accurate, efficient and effective operation of radiation therapy device 10.

Those skilled in the art will appreciate that various adaptations and modifications of the just described preferred embodiments can be configured without departing from the scope and spirit of the invention. For example, while a beam peaking process for an X-Ray beam has been described, those skilled in the art will recognize that embodiments of the present invention may be used to maintain and tune other types of beams, including, for example, photon beams. Further, other types of tuning and maintenance may be performed using embodiments of the present invention. While specific signals have been described as supplying inputs to signal converter 50, those skilled in the art will appreciate that other signals from radiation therapy device 10 may also be input to signal converter 50, allowing an operator to view different signals and effects, thereby allowing a wide variety of diagnostic and maintenance activities. Therefore, it is to be understood that, within the scope of the appended claims, the invention may be practiced other than as specifically described herein.

What is claimed is

1. A method for tuning a radiation therapy device using a treatment processing unit coupled to said radiation therapy device, comprising:
selecting an operational parameter of said radiation therapy device;
configuring a signal converter to receive an output signal from said radiation therapy device, said output signal depending on said operational parameter; and
varying said operational parameter while displaying said output signal.

2. The method of claim 1, further comprising:
continuing said varying until said output signal reaches a desired value.

3. The method of claim 1, wherein said operational parameter includes at least one of: a dose rate; an energy level; an operational mode; an input power frequency; and an input to an electromagnet.

4. The method of claim 1, wherein said output signal includes at least one of: a beam current; an RF reflected power signal; a pulse current; a dose pulse signal; an injection current; and an input power frequency.

5. The method of claim 2, further comprising:
continuing said varying while said radiation therapy device is in a closed loop mode until said output signal reaches said desired value; and
switching said radiation therapy device to an open loop mode; and
comparing said output signal in said closed loop mode with said output signal in said open loop mode.

6. The method of claim 1, wherein said varying and said displaying said output signal are performed on a single display unit of said treatment processing unit.

7. The method of claim 1, wherein said varying and said displaying said output signal are performed in separate windows of a single display unit of said treatment processing unit.

8. The method of claim 1, wherein said varying and said displaying said output signal are performed on separate display monitors of said treatment processing unit.

9. A computer-implemented method for tuning a radiation therapy device from a treatment processing unit, comprising:
selecting an operational parameter of said radiation therapy device;
configuring a signal converter of said treatment processing unit to receive an output signal from said radiation therapy device; and
varying said operational parameter while displaying said output signal, said varying and said displaying performed on a display device of said treatment processing unit.

10. A treatment processing unit, comprising:
a processor;
a memory unit in communication with the processor and storing a program, wherein the processor is operative with the program to forward control data to said radiation therapy device to operate said radiation therapy device;
receive signal data from said radiation therapy device, said signal data varying based on said control data;
convert said signal data into a digital representation of said signal data; and
display said digital representation of said signal data on a display device of said treatment processing unit.

11. The treatment processing unit of claim 10, wherein said processor is further operative with the program to vary said control data forwarded to said radiation therapy device.

12. The treatment processing unit of claim 11, wherein said processor is further operative with the program to continue said varying of said control data, receiving signal data, converting signal data, and displaying said digital representation of said signal data until a desired combination of said control data and said signal data is achieved.

13. The treatment processing unit of claim 12, wherein said processor is further operative with the program to record said desired combination.

14. The treatment processing unit of claim 12, wherein said processor is further operative with the program to update control data in a dosimetry unit based on said desired combination.

15. A radiation therapy system, comprising:
a radiation therapy device; and
a treatment processing unit in communication with said radiation therapy device operable to transmit operational parameters to said radiation therapy device and to receive signal data from said radiation therapy device, said treatment processing unit having a display device configured to display said operational parameters along with said signal data.

16. The radiation therapy system of claim 15, wherein said treatment processing unit further comprises:
a signal converter coupled to receive said signal data and operable to convert said signal data into a digital representation of said signal data.

17. The radiation therapy system of claim 16, wherein said signal converter is coupled to receive said signal data from a beam signal cable coupled to said radiation therapy device.

18. The radiation therapy system of claim 15, wherein said signal converter is coupled to receive at least one of: a beam current; an RF reflected power signal; a pulse current; a dose pulse; an injection current; and an input power frequency.

19. The radiation therapy system of claim 15, wherein said operational parameters include at least one of: a dose rate; an energy level; an operational mode; an input power frequency; and an input to an electromagnet.

20. A maintenance display generated by a treatment processing unit, said display containing input parameter data received from a user and provided to a radiation therapy device and output signal data received from said radiation therapy device and displayed to the user, said treatment processing unit having a memory with computer-executable instructions that implement the user inputs to cause the computer to perform the steps comprising:
(a) forward input parameter data to said radiation therapy device to control the operation of said radiation therapy device;
(b) receive analog output signal data from said radiation therapy device;
(c) convert said analog output signal data to said output signal data; and
(d) display said output signal data on said maintenance display.
